(19) United States
(12) Patent Application Publication
(10) Pub. No.: US 2026/0041538 A1
(43) Pub. Date: Feb. 12, 2026

(54) SHAPE MEMORY ALLOY URETHRAL CONTINENCE DEVICE

(71) Applicant: THE JOHNS HOPKINS UNIVERSITY, Baltimore, MD (US)

(72) Inventors: Andrew Cohen, Baltimore, MD (US); Feng Zhu, Baltimore, MD (US); Guanhao Fu, Baltimore, MD (US); Nathaniel J. Leon, Baltimore, MD (US)

(21) Appl. No.: 19/101,498

(22) PCT Filed: Aug. 7, 2023
(86) PCT No.: PCT/US2023/029616

§ 371 Date: Feb. 5, 2025

Related U.S. Application Data
(60) Provisional application No. 63/395,664, filed on Aug. 5, 2022.

(51) Int. Cl. A61F 2/00; A61L 31/04; A61L 31/10

(52) U.S. Cl. CPC A61F 2/0031; A61L 31/04; A61L 31/10

(57) ABSTRACT

A clamp formed from a shape memory metal promotes and manages urethral continence. A method for insertion of the clamp is also included, herein. The clamp can be surrounded by a sleeve. A power source is used to actuate the clamp. The clamp is positioned around the urethra, and in its resting state the clamp is closed to prevent the flow of urine. When the user wants to release the flow of urine, power is applied to the clamp to heat the shape memory material. Heating the shape memory material, allows the clamp to open and release the flow of urine. When the user wants to stop the flow of urine, the power source is turned off and the clamp returns to its closed position.

### CROSS REFERENCE TO RELATED APPLICATIONS

This application claims the benefit of U.S. Provisional Ser. No. 63/395,664 filed on, Aug. 5, 2022, which is incorporated by reference, herein, in its entirety.

### FIELD OF INVENTION

The present invention relates generally to the field of medical devices. More particularly, the present invention relates to a shape memory alloy device to promote urethral continence and a method of insertion of the device.

### BACKGROUND

Urinary incontinence is the loss of bladder control and the resultant leaking of urine. Urinary incontinence can be the result of everyday habits, an underlying medical condition, or physical problems. In some instances, urinary incontinence can result from a lack of or reduced control over the urethra, such as results from a swollen prostate, prostate cancer, or other conditions. This condition can cause discomfort and embarrassment.

Male urinary incontinence is a serious health concern associated with significant reductions in quality of life. Unfortunately, stress urinary incontinence (SUI) is not often amenable to medical treatment and men turn to surgical means for relief.

Erosion, urethral atrophy, and infection are common results of current generation surgical intervention. These are serious complications that require removal and a return to baseline leakage. Upwards of 30% of patients require operative reintervention within 5 years due to mechanical failures.

A device and method for the promotion and management of urethral continence is therefore needed.

### SUMMARY

The foregoing needs are met, to a great extent, by the present invention wherein in one aspect, a device for promotion of urethral continence includes a clamp having a first arm and a second arm. The first arm has a first end and a second end and wherein the second arm has a first end and a second end. The first end of the first arm is connected to first end of the second arm. The device includes a sleeve. The sleeve is configured to surround the first arm and the second arm of the clamp. The device also includes a closure to hold the second end of the first arm and the second end of the second arm together.

According to an embodiment of the present invention, the clamp is formed from a shape memory material. The sleeve is formed from a biocompatible material. The biocompatible material can take the form of a plastic. The sleeve can include a coating. The coating can take the form of a silicone-based biocompatible coating.

In accordance with another aspect of the present invention, the device includes a power source to apply current to the clamp and increase a temperature of the clamp. The clamp is in a closed position at ambient temperature and opens when the temperature of the clamp is increased. The power source is incorporated into the clamp. Alternately, the power source is positioned separately from the clamp. The power source is coupled to the clamp via a wired connection. In some embodiments, the clamp is actuated wirelessly. The power source is configured to deliver a current of 4.95 Amps to the clamp. The clamp is configured to close via force applied by the sleeve. The clamp is configured to open when the current of 4.95 Amps is applied to the clamp.

In accordance with yet another aspect of the present invention, a device for promotion of urethral continence includes a clamp. The clamp is formed from a shape memory material. The device also includes a sleeve. The sleeve is configured to surround the clamp.

In accordance with still another aspect of the present invention, the device includes a power source to apply current to the clamp and increase a temperature of the clamp. The clamp is in a closed position at ambient temperature and opens when the temperature of the clamp is increased. The sleeve is formed from a biocompatible material. The biocompatible material is a plastic. The sleeve can further include a coating. The coating can take the form of a silicone-based biocompatible coating. A power source applies current to the clamp. The power source can be incorporated into the clamp or can be positioned separately from the clamp. The power source is coupled to the clamp via a wired connection. The clamp is actuated wirelessly. The power source is configured to deliver a current of 4.95 Amps to the clamp. The clamp is configured to close via force applied by the sleeve. The clamp is configured to open when the current of 4.95 Amps is applied to the clamp.

### BRIEF DESCRIPTION OF THE DRAWINGS

Further objectives and advantages will become apparent from a consideration of the description, drawings, and examples.

FIG. 1 illustrates a front view of a urethral continence device clamped around a model urethra, according to an embodiment of the present invention.

FIG. 2A illustrates a side view of a urethral continence device of the present invention.

FIG. 2B illustrates a partially exploded view of the urethral continence device of FIG. 2A, according to an embodiment of the present invention.

FIG. 3 illustrates a graphical view of differential scanning calorimetry (DSC) analysis of the shape memory plate, according to an embodiment of the present invention.

### DETAILED DESCRIPTION

The presently disclosed subject matter will now be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention provides a device for the promotion and management of urethral continence. The device includes a clamp formed from a shape memory material. The clamp can be surrounded by a sleeve. A power source is used to actuate the clamp. The clamp is positioned around the urethra, and in its resting state the clamp is closed to prevent the flow of urine. When the user wants to release the flow of urine, power is applied to the clamp to heat the shape memory material. Heating the shape memory material, allows the clamp to open and release the flow of urine. When the user wants to stop the flow of urine, the power source is turned off and the clamp returns to its closed position.

In some embodiments, the device takes the form of a clamp formed from a shape memory material. One example is a Nitinol-silicone urethral compression device with a ‘deactivated’ configuration which promotes continence at body temperature, and an ‘activated’ mode which will allow free passage of urine with a small rise in device temperature. The unique properties shape memory materials allow shape modulation based on ambient temperature. The shape memory material can be molded to safely occlude the urethra. Using a shape memory material will fundamentally simplify and revolutionize the surgical treatment of stress urinary incontinence, in men. Use of such a device would require only a single incision, would have no mechanical parts prone to breakage, and would be activated with an external device, greatly generalizing applicability. The shape memory material allows the clamp to be closed at ambient temperature and open when heat is applied.

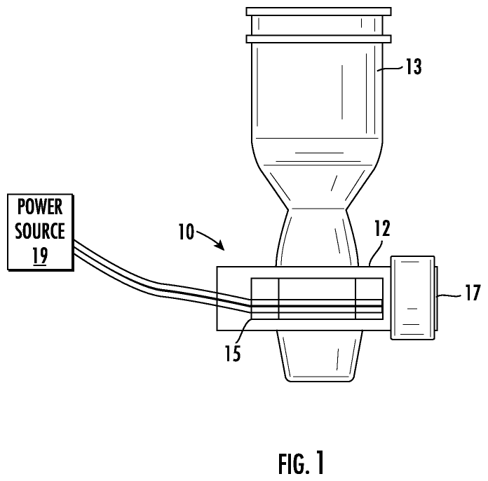

FIG. 1 illustrates a front view of a urethral continence device clamped around a model urethra, according to an embodiment of the present invention. The urethral continence device 10 includes a clamp 12 formed from a shape memory material, such as nitinol. In FIG. 1, the clamp 12 is shown to be clamped around a model urethra 13. The shape memory material allows the clamp to be closed at ambient temperature and open when heat is applied. The clamp 12 is enveloped by a sleeve 15 formed from a biocompatible material, such as an elastic resin. The sleeve 15 also comprises a coating 17. The coating 17 can take the form of any biocompatible coating, but in preferred embodiments the coating takes the form of a biocompatible silicone-based coating. In some embodiments the coating 17 can be applied to the sleeve 15, and in other embodiments the coating 17 could be integrated into a surface of the sleeve 15 at manufacture. Alternately, the coating 17 can be applied or incorporated in any other manner known to or conceivable to one of skill in the art.

In some embodiments, the urethral continence device 10 is wired to connect it to a power source 19 to apply current to change the temperature of the shape memory material for actuating the clamp. In other embodiments, the urethral continence device can also be wireless. The urethral continence device 10 can be actuated with an external device to trigger opening and closing of the clamp 12. In other embodiments, the urethral continence device 10 can include a microprocessor with wireless, Bluetooth, RFID, or other similar capabilities, and can be actuated with an external device, smartphone, or other smart device.

In the exemplary implementation of FIG. 1, the clamp 12 includes a 0.17 mm thick by 5 mm wide plate smooth metal alloy inserted into a 3D printed elastic resin sleeve. The sleeve was printed with FormLabs Elastic 50A V1 resin. Two wires are attached to either end of the bent plate to allow for a power source to be easily connected. It is to be understood that these dimensions, materials, and methods of manufacture are included herein, by way of example. These details are not meant to be considered limiting and any appropriate dimensions, materials, and methods of manufacture known to or conceivable to one of skill in the art are included herein.

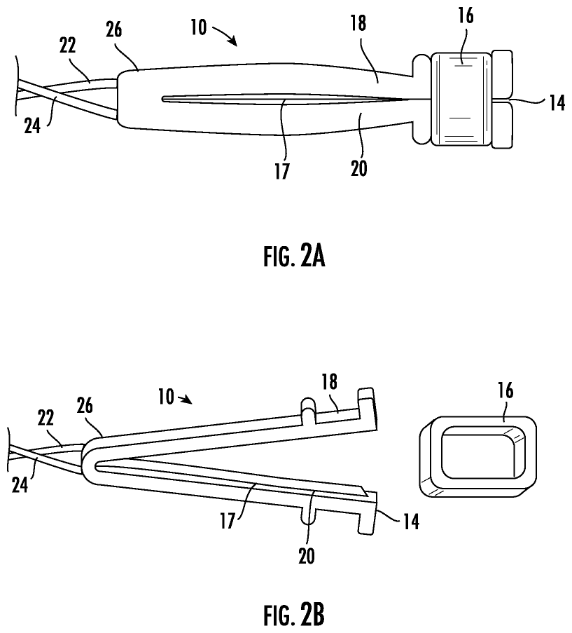

FIG. 2A illustrates a side view of a urethral continence device of the present invention, and FIG. 2B illustrates a partially exploded view of the urethral continence device of FIG. 2A, according to an embodiment of the present invention. As illustrated in FIGS. 2A and 2B, the device 10 can be opened on one end 14 for safe insertion around the urethra and has a band 16 that can be wrapped around the open end 14 to secure it. With the clamp 12 secured by the band 16, the clamp 12 can still be opened to allow for urination while staying in place. To open the clamp 12 within 22 seconds of starting actuation, a current of 4.95 Amps can be run through the plate for that time. After turning off the current, the plate will flatten out and return to the closed position by the force of the elastic resin sleeve within 2 minutes of turning off the power. The band can include a coating 17, which in some embodiments takes the form of a silicone-based biocompatible coating.

The clamp 12 includes a first arm 18 and a second arm 20. The first arm 18 and the second arm 20 are coupled at a second end 26. The first arm 18 and the second arm 20 can be coupled using a fastener, adhesive, pressure fitting, crimping, or any other means of attachment known to or conceivable to one of skill in the art. In other implementations, the first arm 18 and the second arm 20 can be formed from one continuous piece of material that is folded or manipulated to form the first arm 18 and the second arm 20. In the deactivated configuration, the first arm 18 and the second arm 20 of the clamp are in a closed position to clamp the urethra closed. In the activated position, the first arm 18 and the second arm 20 open to allow the passage of urine. In a wired implementation of the device, wires 22 and 24 are coupled to each of the first and second arms 18, 20.

The clamp 12 is inserted through an approximately 3 cm incision between the patient's scrotum and anus with the first arm 18 and the second arm 20 in an open configuration, as illustrated in FIG. 2A. Dissection would commence down through the bulbospongiosus muscle to the corpora spongiosum. An approximately 1.5 cm window on the dorsal surface of the spongiosum would be created, separating the corpora spongiosum from the underlying tunical albuginea of the corpora cavernosa. The clamp 12 in its open configuration allows for insertion around the urethra at this point. The clamp 12 is positioned about the urethra, such that the first arm 18 is on one side of the urethra and the second arm 20 is on another side of the urethra. Then the device is tipped and rotated slightly out of the incision but still around the urethra. This positioning is illustrated in FIG. 1, where the clamp 12 is positioned about the model urethra. Once the clamp 12 is in position, band 16 is used to close the open end 14 of the clamp 12 and bring the first arm 18 and the second arm 20 into a closed position. The band 16 slides into place. Once the clamp is in position any additional hardware can also be placed, such that the patient can actuate the device when it is time to release urine. For instance the wires 22 and 24 are tunneled to a subcutaneous location for connection to a power source. The wound in the perineum is then closed in multiple layers. These insertion steps are included herein by way of example, and any modification or variation on these steps known to or conceivable to one of skill in the art are considered to be included herein.

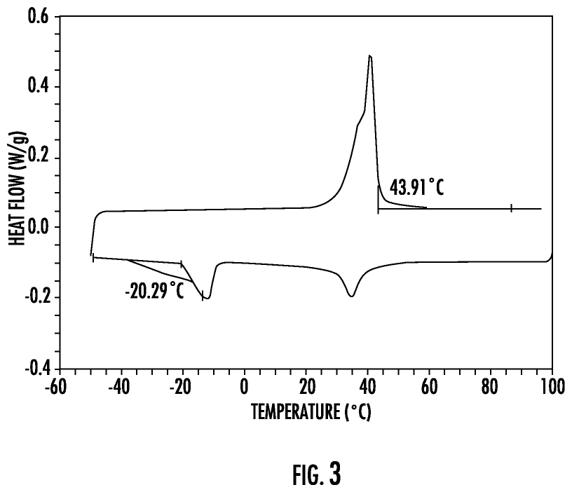

FIG. 3 illustrates a graphical view of differential scanning calorimetry (DSC) analysis of the shape memory plate, according to an embodiment of the present invention. plate, according to an embodiment of the present invention. The DSC test of a sample from the plate used in the final prototype shows an austenite finish of 43.91° C. Even though the plate has an austenite finish higher than 42° C., actuating the plate with 2.1 Volts and 4.95 Amps for 21.43 seconds will open the clamp and will not heat the outside surface of the resin sleeve higher than 42° C. The outside surface temperature of the resin sleeve started at 26.7° C., and after actuation reached 35.6° C. After stopping the current, the sleeve took 2 minutes to fully close on its own. This analysis of the shape memory metal used in the exemplary implementation described above is included herein only by way of example to further illustrate the invention. These criteria are not meant to be considered limiting, and any implementation of the principles of the invention known to or conceivable to one of skill in the art are understood to be included herein.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected.

The above-described embodiments of the invention may be modified or varied, without departing from the invention, as appreciated by those skilled in the art in light of the above teachings. It should also be noted that while the present invention is discussed with reference to its important use for. It is therefore to be understood that, within the scope of the claims and their equivalents, the invention may be practiced otherwise than as specifically described.

1. A device for promotion of urethral continence comprising:
a clamp having a first arm and a second arm, wherein the first arm has a first end and a second end and wherein the second arm has a first end and a second end, and wherein the first end of the first arm is connected to first end of the second arm;
a sleeve, wherein the sleeve is configured to surround the first arm of the clamp and the second arm of the clamp; and,
a closure to hold the second end of the first arm of the clamp and the second end of the second arm of the clamp together.

2. The device of claim 1 wherein the clamp is formed from a shape memory material.

3. The device of claim 1 wherein the sleeve is formed from a biocompatible material.

4. The device of claim 3 wherein the biocompatible material is a plastic.

5. The device of claim 1 further comprising a coating.

6. The device of claim 5 wherein the coating comprises a silicone-based biocompatible coating.

7. The device of claim 1 further comprising a power source to apply current to the clamp.

8. The device of claim 7 wherein the power source is incorporated into the clamp.

9. The device of claim 7 wherein the power source is positioned separately from the clamp.

10. The device of claim 9 wherein the power source is coupled to the clamp via a wired connection.

11. The device of claim 1 wherein the clamp is in a closed position at ambient temperature and opens when the temperature of the clamp is increased.

12. The device of claim 1 wherein the clamp is actuated wirelessly.

13. The device of claim 7 wherein the power source is configured to deliver a current of 4.95 Amps to the clamp.

14. The device of claim 13 wherein the clamp is configured to close via force applied by the sleeve.

15. The device of claim 13 wherein the clamp is configured to open when the current of 4.95 Amps is applied to the clamp.

16. A device for promotion of urethral continence comprising:
a clamp, wherein the clamp is formed from a shape memory material; and
a sleeve, wherein the sleeve is configured to surround the clamp.

17. The device of claim 16 wherein the sleeve is formed from a biocompatible material.

18. The device of claim 17 wherein the biocompatible material is a plastic.

19. The device of claim 16 further comprising a coating.

20. The device of claim 19 wherein the coating comprises a silicone-based biocompatible coating.

21. The device of claim 16 further comprising a power source to apply current to the clamp.

22. The device of claim 21 wherein the power source is incorporated into the clamp.

23. The device of claim 21 wherein the power source is positioned separately from the clamp.

24. The device of claim 21 wherein the power source is coupled to the clamp via a wired connection.

25. The device of claim 16 wherein the clamp is in a closed position at ambient temperature and opens when the temperature of the clamp is increased.

26. The device of claim 16 wherein the clamp is actuated wirelessly.

27. The device of claim 21 wherein the power source is configured to deliver a current of 4.95 Amps to the clamp.

28. The device of claim 27 wherein the clamp is configured to close via force applied by the sleeve.

29. The device of claim 27 wherein the clamp is configured to open when the current of 4.95 Amps is applied to the clamp.
